(19) United States
(12) Patent Application Publication
(10) Pub. No.: US 2012/0263739 A1
(43) Pub. Date: Oct. 18, 2012

(54) ANTI INTEGRIN ANTIBODIES LINKED TO NANOPARTICLES LOADED WITH CHEMOTHERAPEUTIC AGENTS

(75) Inventors: Klaus Langer, Muenster (DE); Marion Anhorn, Frankfurt am Main (DE); Joerg Kreuter, Frankfurt (DE); Florian Rothweiler, Frankfurt am Main (DE); Hagen von Briesen, Huenstetten (DE); Sylvia Wagner, Neunkirchen (DE); Martin Michaelis, Whitstable (GB); Jindrich Cinatl, Offenbach (DE)

(73) Assignee: MERCK PATENT GMBH, DARMSTADT (DE)

(21) Appl. No.: 13/509,492

(22) PCT Filed: Oct. 21, 2010
(86) PCT No.: PCT/EP10/06443

§ 371 Date: Jun. 29, 2012

(30) Foreign Application Priority Data
Nov. 13, 2009 (EP) 09014206.8

(51) Int. Cl. C07K 19/00; A61P 35/00; A61K 39/44

(52) U.S. Cl. 424/178.1; 530/391.7; 530/363; 530/391.9

(57) ABSTRACT

The invention relates to anti-integrin antibodies which are covalently linked to nanoparticles, wherein these nanoparticles were prior loaded with chemotherapeutic/cytotoxic agents. The antibody-chemotherapeutic agent-nanoparticle conjugates according to the invention, especially wherein the antibody is MAb DI17E6 and the cytotoxic agent is doxorubicin show a significant increase of tumor cell toxicity.

### TECHNICAL FIELD OF THE INVENTION

The invention relates to anti-integrin antibodies which are covalently linked to nanoparticles. These nanoparticles are preferably loaded with or bound to chemotherapeutic agents. The antibody-chemotherapeutic agent-nanoparticle conjugates show a significant increase of tumor cell toxicity. The invention is especially directed to such antibody conjugates, wherein the antibody is an integrin inhibitor, preferably an av integrin blocking antibody and the nanoparticle is a serum albumin nanoparticle. The antibody nanoparticle conjugates of this invention can be used for tumor therapy. Therefore, antibody-coupled human serum albumin nanoparticles represent a potential delivery system for targeted drug transport into tumor receptor-positive or tumor receptor expressing cells.

### TECHNICAL BACKGROUND OF THE INVENTION

In the last years new strategies for cancer treatment based on drug loaded nanoparticulate formulations emerged in cancer research.

Nanoparticles represent promising drug carriers especially for specific transport of anti-cancer drugs to the tumor site. Nanoparticles show a high drug loading efficiency with minor drug leakage, a good storage stability and may circumvent cancer cell multidrug resistance [Cho K, Wang X, Nie S, Chen Z G, Shin D M.; Clin Cancer Res 2008; 14(5):1310-13161. Nanoparticles made of human serum albumin (HSA) offer several specific advantages [Weber C, Coester C, Kreuter J, Langer K.; Int J Pharm 2000; 194(1):91-102]: HSA is well tolerated and HSA nanoparticles are biodegradable. HSA nanoparticle preparation is easy and reproducible [Langer K, Balthasar S, Vogel V, Dinauer N, von Briesen H, Schubert D.; Int J Pharm 2003; 257(1-2):169-180] and covalent derivatisation of nanoparticles with drug targeting ligands is possible, due to the presence of functional groups on the surfaces of the nanoparticles [Nobs L, Buchegger F, Gurny R, Allemann E.; J Pharm Sci 2004; 93(8):1980-1992; Wartlick H, Michaelis K, Balthasar S, Strebhardt K, Kreuter J, Langer K.; J Drug Target 2004; 12(7):461-471; Dinauer N, Balthasar S, Weber C, Kreuter J, Langer K, von Briesen H.; Biomaterials 2005; 26(29):5898-5906; Steinhauser I, Spänkuch B, Strebhardt K, Langer K.; Biomaterials 2006; 27(28):4975-4983].

The enrichment of the nanoparticles in tumor tissue might occur by passive or active targeting mechanisms. Passive targeting results from the “Enhanced Permeability and Retention (EPR) effect” characterized by enhanced accumulation of nanoparticulate systems in tumors due to leaky tumor vasculature in combination with poor lymphatic drainage [Maeda H, Wu J, Sawa T, Matsumura Y, Hori K.; J Control Release 2000; 65(1-2):271-284]. Especially, long circulating nanoparticles with poly (ethylene) glycol (PEG) modifications on their surface are known to show passive tumor targeting [Greenwald R B;. J Control Release 2001; 74(1-3):159-171].

Coupling of tumor-specific ligands on the surface of drug carrier systems results in active drug targeting. Monoclonal antibodies (mAbs) offer great potential as drug targeting ligands [Adams G P, Weiner L M.; Nat Biotechnol 2005; 23(9):1147-1157].

Cancer cells from various entities have been reported to express high levels of integrin αvβ3 [Albelda S M, Mette S A, Elder D E, Stewart R, Damjanovich L, Herlyn M, et al.; Cancer Res 1990; 50(20):6757-6764; Pijuan-Thompson V, Gladson C L.; J Biol Chem 1997; 272(5):2736-2743; Rabb H, Barroso-Vicens E, Adams R, Pow-Sang J, Ramirez G; Am J Nephrol 1996; 16(5):402-408; Liapis H, Adler L M, Wick M R, Rader J S.; Hum Pathol 1997; 28(4):443-449; Bello L, Zhang J, Nikas D C, Strasser J F, Villani R M, Cheresh D A, et al.; Neurosurgery 2000; 47(5):1185-1195; Gladson C L.; J Neuropathol Exp Neurol 1996; 55(11):1143-1149; Gladson C L, Hancock S, Arnold M M, Faye-Petersen O M, Castleberry R P, Kelly D R.; Am J Pathol 1996; 148(5):1423-1434; Patey M, Delemer B, Bellon G, Martiny L, Pluot M, Haye B.; J Clin Pathol 1999; 52(12):895-900; Ritter M R, Dorrell M I, Edmonds J, Friedlander S F, Friedlander M.; Proc Natl Acad Sci USA 2002; 99(11):7455-7460.]

αvβ3 integrin is a receptor for extracellular matrix (ECM) ligands such as vitronectin, fibronectin, fibrinogen, laminin and is also called “vitronectin receptor”. Most tissues and cell types are characterized by low αvβ3 integrin levels or absence of αvβ3 integrin expession. However, it is overexpressed on endothelial cells and smooth muscle cells after activation by cytokines, especially in blood vessels from granulation tissues and tumors [Eliceiri B P, Cheresh D A.; J Clin Invest 1999; 103(9):1227-1230]. Therefore, it has an important function during angiogenesis. αvβ3 integrin is involved in melanoma growth in in vivo-models. αvβ3 inhibitors block the angiogenesis and the tumor growth [Mitjans F, Sander D, Adan J, Sutter A, Martinez J M, Jaggle C S, et al.; J Cell Sci 1995; 108 (Pt 8):2825-2838; Mitjans F, Meyer T, Fittschen C, Goodman S, Jonczyk A, Marshall J F, et al.; Int J Cancer 2000; 87(5):716-723]. Furthermore, in some cancers such as breast cancer or melanoma, αvβ3 expression appears to correlate with the aggressiveness of the disease [Brooks P C, Stromblad S, Klemke R, Visscher D, Sarkar F H, Cheresh D A.; J Clin Invest 1995; 96(4):1815-1822; Felding-Habermann B, Mueller B M, Romerdahl C A, Cheresh D A.; J Clin Invest 1992; 89(6):2018-2022].

Antagonists of integrin αvβ3 not only prevent the growth of tumor-associated blood vessels but also provoke the regression of established tumors in vivo. Various antibodies, antagonists, and small inhibitory molecules have been developed as potential antiangiogenic strategies, implicating that the integrin αvβ3 may be a potential target on endothelial cells for specific antiangiogenic therapy, decreasing tumor growth and neovascularization, as well as increasing the tumor apoptotic index [Brooks P C, Montgomery A M, Rosenfeld M, Reisfeld R A, Hu T, Klier G, et al.; Cell 1994; 79(7):1157-1164; Petitclerc E, Stromblad S, von Schalscha T L, Mitjans F, Piulats J, Montgomery A M, et al.; Cancer Res 1999; 59(11):2724-2730].

Monoclonal mouse antibody 17E6 inhibits specifically the αv-integrin subunit of human integrin receptor bearing cells. The mouse IgG1 antibody is described, for example by Mitjans et al. (1995; J. Cell Sci. 108, 2825) and patents U.S. Pat. No. 5,985,278 and EP 719 859. Murine 17E6 was generated from mice immunized with purified and Sepharose-immobilized human αvβ3. Spleen lymphocytes from immunized mice were fused with murine myeloma cells and one of the resulting hybridoma clones produced monoclonal antibody 17E6. DI-17E6 is an antibody having the biological characteristics of the monoclonal mouse antibody 17E6 but with improved properties above all with respect to immunogenicity in humans. Properties of DI17E6 and its complete variable and constant amino acid sequence of this modified antibody is presented in PCT/EP2008/005852. The antibody has the following sequence:

(i) variable and constant light chain sequences (SEQ ID No. 1):

DIQMTQSPSSLSASVGDRVTITCRASQDISNYLAWYQQKPGKAPKLLIY
YTSKIHSGVPSRFSGSGSGTDYTFTISSLQPEDIATYYCQQGNTFPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

and

(ii) variable and constant heavy chain sequences (SEQ ID No. 2):

QVQLQQSGGELAKPGASVKVSCKASGYTFSSFWMHWVRQAPGQGLEWIG
YINPRSGYTEYNEIFRDKATMTTDTSTSTAYMELSSLRSEDTAVYYCAS
FLGRGAMDYWGQGTTVTVSSASTKGPSVFPLAPCSRSTSESTAALGCLV
KDYFPEPVTVSWNSGALTSGVHTFPAVLQSSGLYSLSSVVTVPSSNFGT
QTYTCNVDHKPSNTKVDKTVEPKSSDKTHTCPPCPAPPVAGPSVFLFPP
KPKDTLMISRTPEVTCVVVDVSHEDPEVQFNWYVDGVEVHNAKTKPREE
QAQSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYK
TTPPMLDSDGSFFLYSKLTVDKSRWQQGNVFSCSVMHEALHNHYTQKSL
SLSPGK.

In vitro these antibodies block cell adhesion and migration and it induces cell detachment from vitronectin coated surfaces. In endothelial cells, it also induces apoptosis. Effects are increased in combination with chemotherapy. In vivo, DI17E6 blocks growth of melanomas and other tumors and growth factor-induced angiogenesis. Therefore, 17E6 as well as DI17E6 mAb may interfere both directly with tumor cells and with tumor angiogenesis [Mitjans F, Sander D, Adan J, Sutter A, Martinez J M, Jaggle C S, et al.; J Cell Sci 1995; 108 (Pt 8):2825-2838; Mitjans F, Meyer T, Fittschen C, Goodman S, Jonczyk A, Marshall J F, et al.; Int J Cancer 2000; 87(5):716-723].

Other anti-αvβ3 antibodies are for example, vitaxin or LM609.

Chemotherapeutic agents are generally used in the treatment of cancer diseases. It was shown they show extraordinary tumor cell toxicity if applied together or at least in conjunction with antibody administration. Most of the known and marketed anti-tumor antibodies are effective only in a combination treatment with chemotherapeutic agents, such as cisplatin, doxorubicin or irinotecan.

Therefore, the problem of the invention to be solved is to provide an anti-integrin, preferably an anti-aᵥ antibody which is linked directly or indirectly to the surface of a nanoparticle in order to enhance the efficacy of the antibody in a therapy preferably a tumor therapy in conjunction with chemotherapy.

### SUMMARY OF THE INVENTION

It was found that if antibodies are linked to a protein based nanoparticle, preferably to a serum albumin nanoparticle, the efficacy of the antibody in context with anti-tumor activity can be generally enhanced when treatment is combined with chemotherapy by chemotherapeutic agents. Surprisingly, this effect is extraordinaire, when the protein-nanoparticles to which the respective antibody is linked are loaded with the chemotherapeutic agent that is intended for use in an chemotherapeutic agent/antibody combination therapy. The cytotoxicity of the protein nanoparticle loaded with a chemotherapeutic agent and linked covalently to an anti-tumor antibody is higher as a respective nanoparticle loaded with the chemotherapeutic agent alone or with the antibody alone. The cytotoxic effect of the complete conjugate is even enhanced versus the combination of free chemotherapeutic agent and free anti-tumor antibody.

The invention is especially directed to respective conjugates, wherein for example Mab 17E6 or its deimmunized version DI17E6 is coupled to the surface of doxorubicin-loaded HSA nanoparticles. After coupling, the biological activity of DI17E6 was indicated by adhesion studies to αvβ3-positive cells and induction of detachment of αvβ3-positive cells from vitronectin-coated surfaces. Moreover, doxorubicin-modified DI17E6 nanoparticles induce more enhanced anti-cancer effects in αvβ3-positive cancer cells than free doxorubicin and free antibody.

According to the invention the effect can be shown also for anti-tumor antibodies other than 17E6 or DI17E6, such other anti-integrin antibodies, as well as for chemotherapeutic agents other than doxorubicin, such as irinotecan or cisplatin.

The invention is preferably directed to HSA nanoparticles

A major goal in nanotechnology research is an active targeting of nanoparticulate carriers with the advantage of an efficient accumulation of drugs in tumor tissue to achieve higher drug levels in target cells. Therefore, drug targeting ligands of monoclonal antibody origin are often used. This invention describes the preparation of specific human serum albumin based nanoparticles loaded with a chemotherapeutic agent, such as doxorubicin. By coupling of, for example, DI17E6, a monoclonal antibody directed against αv integrins to the nanoparticle surface, a specific targeting of αvβ3 integrin expressing cancer cells is possible.

According to the invention a covalent binding between antibody and nanoparticle surface thiolation of the antibody is necessary. The tendency of dimerization of the thiolated antibodies but also the efficiency of sulfhydryl group introduction within the antibody has to be taken into account. The longer the thiolation time and the higher the molar excess of the thiolation reagent 2-iminothiolane, the larger is the excess of antibody dimerization. This dimerization process resulted probably by disulfide bond formation between two antibody molecules.

The quantification of the introduced thiol groups by using 2-iminothiolane at, for example, a 50 or 100 fold molar excess at incubation times of 2 and 5 h show that at least an 50 fold molar excess of 2-iminothiolane is necessary for effective thiolation. The longer the incubation time and the larger the molar excess of the thiolation reagent the more thiol groups/antibody can be introduced within the protein molecules. On the basis of our results, with a compromise of thiolation efficiency and dimerization behaviour, the parameters of our standard protocol are fixed to 2 h and 50 fold molar excess of 2-iminothiolane.

Due to the IgG origin of the DI17E6 antibody it can be shown that DI17E6 binds to nanoparticle surface with the gold anti-human IgG antibody reaction in the SEM. The nanoparticles are shown as grey spheres in the SEM pictures in a range of 150-220 nm. The DI17E6 coupling on the nanoparticle surface was indirectly shown by the reflections of the electron beam on the gold surface.

The invention demonstrates the specific cellular binding and cellular uptake of the HAS nanoparticles modified with different anti-integrin antibodies, such as αv-specific DI17E6 on αvβ3 integrin positive melanoma cells M21. In contrast, no specific binding is detectable after incubation on αv-defective melanoma cells M21L. The loading of the nanoparticles with the cytostatic drug doxorubicin has no influence on this specificity. The control nanoparticles with unspecific mAb IgG on surface show also an unspecific cellular binding and no intracellular uptake, they just stuck on the outer cell membrane.

The biological activity of the antibody, such as DI17E6, is preserved during nanoparticle preparation shown by the cell attachment and detachment assays. In case of DI17E6, both assays are based on the fact that the main cell attachment on vitronectin coated surfaces is done by αvβ3 integrins. The αvβ3 integrins are also called vitronectin receptor. Therefore, an inhibition of the αvβ3 integrins leads to a detachment of already attached cells or inhibits the attachment of cells. DI17E6 as well as DI17E6-modified nanoparticulate formulations are able to block the αvβ3 integrin sites on αvβ3 positive melanoma cells M21 and to inhibit the attachment of the cells on vitronectin coated surfaces. Furthermore, they can detach already attached cells whereas nanoparticulate formulations with a control antibody have just little influence on cell attachment. Similar observations can be made with other antibodies within respective approaches.

A parallel detachment kinetic study of the different nanoparticulate formulations or free cytotoxic agent, such as doxorubicin confirms the cell detachment assay results. In case of DI17E6 and doxorubicin, cell detachment is induced by the NP-DI17E6 and the NP-Dox-DI17E6, but the doxorubicin loaded nanoparticles seem to be more efficient. In addition, a more surprising result is the faster induction of cell death by the doxorubicin containing nanoparticles than by free doxorubicin.

The IC-50 values of the MTT assay also support these findings of a higher cytotoxicity of nanoparticulate bound doxorubicin than free cytotoxic agent. A lower concentration of NP-CA-MAb (wherein NP is nanoparticle, CA is cytotoxic or chemotherapeutic agent and Mab is monoclonal antibody), such as NP-Dox-DI17E6 (wherein Dox is doxorubicin) is needed to decrease cell viability than of free cytotoxix agent to induce the same effect. The specific DI17E6 modified doxorubicin loaded nanoparticles seem to be better in cellular doxorubicin transport than free doxorubicin. Due to the ineffectiveness of the DI17E6 modified nanoparticles after incubation on αv-defective melanoma cells M21L and the effectiveness after incubation on αvβ3 positive melanoma cells M21 the specificity of the NP-Dox-DI17E6 can be verified. The IgG modified nanoparticles were ineffective on both cellular systems, the αvβ3 positive melanoma cells M21 and the αv-defective melanoma cells M21L.

The unspecific uptake of unmodified nanoparticles by cancer cells is known but not as effective as with ligand modified nanoparticles, as shown by NP-Dox. In summary, the invention provides an antibody specific/chemotherapeutic agent loaded nanoparticle drug targeting system, preferably a DI17E6 based αv-specific, doxorubicin loaded nanoparticulate drug targeting system, which is more efficient than the free chemotherapeutic/cytotoxix agent and unmodified nanoparticles.

Strategies to specifically transport cytotoxic drugs into tumor cells in order to increase anti-cancer effects and minimize toxic side-effects are of high interest. Many nanoparticle formulations have been investigated in this context (for review see Haley et al., lit. cited). For example, there are FDA approved liposomal doxorubicin encapsulations (Doxil®/Caelyx® and Myocet™) where the anthracycline pharmacokinetics are changed and cardiac risk is decreased [Working P K, Newman M S, Sullivan T, Yarrington J.; J Pharmacol Exp Ther 1999; 289(2):1128-1133; Waterhouse D N, Tardi P G, Mayer L D, Bally M B.; Drug Saf 2001; 24(12):903-920; Gabizon A, Shmeeda H, Barenholz Y.; Clin Pharmacokinet 2003; 42(5):419-436;O'Brien M E, Wigler N, Inbar M, Rosso R, Grischke E, Santoro A, et al.; Ann Oncol 2004; 15(3):440-449].

A further example is the first HSA-based nanoparticle formulation, Abraxane®, approved by the FDA in 2005. These nanoparticles contain the cytostatic drug paclitaxel. Due to the poor solubility of paclitaxel in water, there are a variety of advantages for nanoparticulate-bound paclitaxel like increased intratumoral concentrations, higher doses of delivered paclitaxel and decreased infusion time without premedication [Gradishar W J, Tjulandin S, Davidson N, Shaw H, Desai N, Bhar P, et al.; J Clin Oncol 2005; 23(31):7794-7803; Desai N, Trieu V, Yao Z, Louie L, Ci S, Yang A, et al.; Clin Cancer Res 2006; 12(4)1317-1324].

Here, The invention provides a nanoparticle system that specifically targets αv-integrins and holds potential to target tumor cells that show high expression of αv-integrins and/or inhibit angiogenesis by targeting of endothelial cells.

The invention provides specifically the preparation of target-specific human serum albumin nanoparticles loaded with the cytostatic drug doxorubicin. By the use of DI17E6, a monoclonal antibody directed against αv integrins, for covalent coupling on nanoparticle surface, the specific cellular binding and cellular uptake of DI17E6-modified HSA-nanoparticles on αvβ3 integrin positive melanoma cells can be shown. The biological activity of the DI17E6 antibody is preserved during nanoparticle preparation shown by two biological assays, the cell attachment and detachment assay. The drug loading of this nanoparticulate formulation has no influence on cell detachment assay. Even more, the cell detachment is more efficient in case of cell incubation with drug loaded nanoparticles, compared to cell incubation with unloaded nanoparticles. Furthermore, this drug loaded nanoparticulate formulation induces faster cell death than free doxorubicin. This finding of a higher cytotoxicity of the drug loaded specific nanoparticles compared to the free doxorubicin is supported by a cell viability assay.

In conclusion, the invention provides drug targeting system based on nanoparticles, preferably HAS nanoparticles loaded with a cytotoxic/chemotherapeutic agent to which an anti-integrin receptor antibody, preferably an anti-av antibody, such as DI17E6 is covalently coupled This system is more efficient than the free cytotoxic agent. The combination of specific targeting with drug loading in these nanoparticulate formulations leads to an improvement of cancer therapy. As mentioned above, DI17E6 with its bi-specific properties, on the one hand to block melanoma growth and on the other hand to inhibit angiogenesis, is a promising mAb for cancer therapy. Thus, not only the free DI17E6 but also the DI17E6 modified and drug loaded nanoparticles can act as double-edged sword in tumor therapy.

In summary, the invention is directed to:
an anti-integrin antibody nanoparticle conjugate, obtained by linking covalently an anti-integrin antibody or a biologically active fragment thereof to the surface of a protein-nanoparticle which was prior treated with a chemotherapeutic agent;
a respective antibody nanoparticle conjugate, wherein the chemotherapeutic agent was loaded by adsorption to the protein-nanoparticle;
a respective antibody nanoparticle conjugate, wherein the protein nanoparticle is of human serum albumin (HSA) or bovine serum albumin (BSA);
a respective antibody nanoparticle conjugate, wherein the particle diameter of the untreated protein-nanoparticles is between 150 and 250 nm, preferably between 160 and 190 nm:
a respective antibody nanoparticle conjugate, wherein the particle diameter of the protein-nanoparticles treated with a chemotherapeutic agent is between 300 and 400 nm, preferably between 350 and 390 nm;
a respective antibody nanoparticle conjugate, wherein the antibody was linked directly or by a linker to the protein-nanoparticle via a sulfhydryl group introduced into the antibody molecule;
a respective antibody nanoparticle conjugate, wherein the chemotherapeutic agent treated with said protein-nanoparticle is selected from the group consisting of: cisplatin, doxorubicin, gemcitabine, docetaxel, paclitaxel, bleomycin and irinotecan;
a respective nanoparticle conjugate, wherein the antibody linked covalently to said protein-nanoparticle is selected from the group LM609, vitaxin, and 17E6 and variants thereof;
a respective antibody nanoparticle conjugate, wherein the protein-nanoparticle is HSA that is loaded with doxorubicin and the antibody linked covalently to this particle is 17E6 or DI17E6;
a pharmaceutical composition comprising an antibody nanoparticle conjugate as specified above in an pharmacologically effective amount optionally together with a carrier, eluent or recipient;
the use of an antibody nanoparticle conjugate as specified above for the manufacture of a medicament for the treatment of cancer diseases;
an antibody nanoparticle conjugate as specified above for use in the treatment of tumor diseases.

The HSA nanoparticles obtained according the invention loaded with a chemotherapeutic/cytotoxic agent and linked covalently to an anti-integrin, especially anti-av antibody show cell death already after 10 h in a cell attachment/detachment assay comprising cells bearing integrin receptors to which the antibody specifically binds.

Respective HSA nanoparticles according the invention loaded with a chemotherapeutic/cytotoxic agent and linked to an antibody show cell death after 20 h in said cell attachment/detachment wherein the antibody is not an anti-integrin antibody and the cells does not comprise integrin receptors to which the antibody can bind (IgG).

The free cytotoxic agent shows cell death in such a system after around 17 h.

In such a system nanoparticlex which were not preloaded with the cytotoxic compound but linked to an anti-integrin antibody show no cell death as well as free anti-integrin antibody and cells not treated at all.

Consequently, the antibody nanoparticle conjugates according to the invention lead to a cell death in a synergistic manner.

### DETAILS OF THE INVENTION

Nanoparticle preparation: In order to attach DI17E6 to doxorubicin-loaded HSA nanoparticles, a heterobifuctional NHS-PEG-Mal linker was used, which on the one hand reacts with the amino groups on the surface of the HSA nanoparticles and on the other hand has the potential to react with sulfhydryl groups introduced into the antibody DI17E6.

Thiolation of DI17E6: The introduction of thiol groups to antibodies bears the risk of oxidative disulfide bridge formation leading to dimers or even higher oligomers [Steinhauser I, Spänkuch B, Strebhardt K, Langer K.; Biomaterials 2006; 27(28):4975-4983]. Therefore, fomation of dimers and oligomers is evaluated by size exclusion chromatography (SEC) after incubation periods of 2, 5, 16, and 24 h with 2-iminothiolane. Results show that with increasing thiolation time and molar excess of 2-iminothiolane the retention time of the antibody in the chromatograms is slightly prolonged (FIG. 1A). Additionally, the peak heights decreased and the peaks broadened. Using a 50 molar excess of 2-iminothiolane and an incubation time of 2 h the resulting chromatogram shows an additional peak with a shorter retention time. Molecular weight calibration of SEC reveals that this peak represents a compound with twice the molecular weight of the original antibody. With longer incubation times (5, 16, 24 h) this dimer peak enlarges and the original peak broadens indicating an increase in disulfide bridge formation. This observation is more pronounced with a 100-fold excess of 2-iminothiolane (FIG. 1B).

The number of thiol groups introduced per antibody is quantified by disulfide binding with 5,5′-dithio-bis-2(nitro-benzoic acid) (Ellman's reagent). Since prolonged incubation times have resulted in an enhanced formation of di- and oligomers, DI17E6 is incubated with 2-iminothiolane with a 5 fold, 10 fold, 50 fold, and 100 fold molar excess for 2 h or 5 h. Higher molar excess and/or longer incubation times increase the number of thiol groups per antibody (FIG. 2). Using an incubation time of 2 h the 50 fold molar excess leads to 0.64±0.15 thiol groups/antibody whereas the 100 fold molar excess leads to 1.22±0.09 thiol groups/antibody. After a 5 h incubation period, 50 fold molar excess shows 1.2±0.29 and 100 molar excess 2.9±0.12 thiol groups/antibody.

Preparation of HSA nanoparticles: HSA nanoparticles are prepared by desolvation and are stabilized by glutaraldehyde with a stoichiometric crosslinking of 100% of the particle matrix. The nanoparticles are activated with a heterobifunctional poly(ethylene glycol)-α-maleimide-ω-NHS ester (NHS-PEG5000-Mal) or a monofunctional succinimidyl ester of methoxy poly(ethylene glycol) propionic acid (mPEG5000-SPA), respectively. In the first case the heterobifunctional crosslinker leads to a covalent linkage between antibody and nanoparticle. In the second case, only an adsorptive binding between antibody and nanoparticle is expected because of the non-reactive methoxy group at the end of the poly(ethylene) glycol chain.

The results of the physico-chemical characterization are presented in Table 1 for the unloaded and in Table 2 for the doxorubicin-loaded nanoparticles. The unloaded particles are characterized by a particle diameter of 140 to 190 nm whereas the drug loaded particles show a much higher size in the rage of 350-400 nm. The polydispersity of all nanoparticles ranged between 0.01. This indicates a monodisperse particle size distribution independent whether the particles were drug loaded or surface modified.

The doxorubicin loading of the drug loaded particles is 55-60 pg/mg. Covalent linkage of DI17E6 to the particle surface can be achieved with 14-18 μg antibody/mg nanoparticle for the unloaded particles (NP-DI17E6) and 11-20 μg DI17E6/mg nanoparticle for the particles loaded with doxorubicin (NP-Dox-DI17E6). With the control antibody IgG similar results can be obtained:

Unloaded nanoparticles show a surface modification of 16-18 μg antibody/mg nanoparticle (NP-IgG) whereas drug entrapped particles result in a binding of 15-20 μg IgG/mg nanoparticle (NP-Dox-IgG) on their surface. Only a small amount of antibody is adsorptively attached to the surface of the nanoparticles of unloaded or doxorubicin-loaded nanoparticles. The amount ranged from 2-3 μg/mg (unloaded particles) to 0.1-0.5 μg/mg (doxorubicin loaded particles) for DI17E6 and from 4-8 μg/mg (unloaded particles) to 2-3.5 μg/mg (doxorubicin loaded particles) for IgG.

It can be noticed, that IgG show a higher tendency of adsorptive binding than DI17E6. Moreover, the low antibody adsorption to the nanoparticle surface indicates that the majority of the antibody molecules are covalently attached to the particle surface by the heterobifunctional PEG spacer. For cell culture experiments only the samples with covalent linkage of the antibodies are used.

Antibody visualization on nanoparticle surfaces: DI17E6 is a monoclonal antibody of IgG origin. Therefore, a reaction with the 18 nm colloidal gold anti-human IgG antibody was possible. The nanoparticles are recognized as grey spheres in the scanning electron microscope (SEM) pictures (FIG. 3) in a range of 200 nm. Small white spheres were shown on the surface of nanoparticles with DI17E6 coupling (FIGS. 3A and B) whereas nothing is recognized on the surface of nanoparticles without antibody coupling (FIG. 3C). The small white spheres are reflections of the electron beam on the surface of the gold-labeled samples in the SEM.

Cellular binding: αvβ3 integrin-positive melanoma cells M21 and αv-negative melanoma cells M21L are incubated with DI17E6-coupled nanoparticles (NP-DI17E6) or nanoparticles coupled to an unspecific control mAb IgG (NP-IgG). As shown in FIG. 4A, NPDI17E6 shows a higher binding to M21 cells than NP-IgG. In M21L cells a comparable binding of NP-DI17E6 and NP-IgG is observed, which was reduced compared to M21 cells (FIG. 4B). Doxorubicin incorporation does not affect nanoparticle binding. NP-Dox-DI17E6 shows high binding to M21 cells whereas NPDox-IgG shows low binding to these cells M21 (FIG. 4C). Both nanoparticle preparations show low binding to M21L cells (FIG. 4D).

Cellular uptake and intracellular distribution: The cellular uptake and intracellular distribution of these nanoparticulate formulations are shown by confocal laser scanning microscopy (CLSM). αvβ3 integrin-positive M21 melanoma cells are incubated with NP-Dox-DI17E6, with NP-Dox-IgG, or free Doxorubicin (FIG. 5). Only few NP-Dox-IgG are detected at the outer part of the M21 cell membranes (FIG. 5C), whereas NP-Dox-DI17E6 reaches the inner part of the cells (FIG. 5D, 6). Red doxorubicin fluorescence can be detected after incubation with NP-Dox-DI17E6 (FIG. 5D) as well as after incubation with free doxorubicin (FIG. 5B). FIG. 6 demonstrates the intracellular uptake of the NPDox-DI17E6 in a higher magnification. The overlay of the different fluorescence channels (FIG. 6B-D) verifies the intracellular uptake of NP-Dox-DI17E6 (FIG. 6A). Furthermore, M21 cells incubated with NP-Dox-DI17E6 are optically sliced in a stack of 1 μm thickness each by confocal laser scanning microscopy to prove the intracellular uptake. The picture series is displayed as a gallery (FIG. 7).

Cell attachment/cell detachment: Cellular attachment to vitronectin-coated surfaces is mainly mediated by αvβ3 integrins, the so-called vitronectin receptors. αvβ3 integrin inhibition may lead to a detachment of already attached cells or inhibits the attachment of cells. DI17E6 inhibits the attachment of the M21 cells to vitronectin coated surfaces (FIG. 8). Nanoparticulate formulations with DI17E6 on the particle surface inhibits also the M21 cell attachment to vitronectin whereas nanoparticulate formulations with a control antibody just have a minor influence on cell attachment (FIG. 8).

In the detachment assay a slightly higher DI17E6 concentration is needed for cell detachment than in the attachment assay for attachment inhibition (4 ng/μl and 10 ng/μl respectively compared to 2 ng/μl). However, cell detachment of αvβ3 positive melanoma cells M21 from vitronectin coated surfaces is also possible with NP-DI17E6 as well as with free DI17E6 (FIG. 9). Furthermore, NP-Dox-DI17E6 show the same detachment efficiency (FIG. 9).

A parallel detachment kinetic study of the different nanoparticulate formulations or free doxorubicin confirms the cell detachment assay. In this study detachment is observed by transmitted light time lapse microscopy over a period of 1-2 d. Pictures were done every 7 minutes. The detachment time of the cells is measured. Cell detachment induced by the NP-DI17E6 nanoparticles occurs between 2-22 h (Table 3) whereas the doxorubicin containing nanoparticles NP-Dox-DI17E6 are more efficient, inducing complete detachment within the first 3 h (Table 3). Control nanoparticles with IgG modification NP-Dox-IgG show no cellular detachment (Table 3). In addition, a further advantage of the DI17E6 modified doxorubicin containing nanoparticles is observed: these nanoparticles induce cell death within 10 h, which is faster than by free doxorubicin incubation. In this case the cell death occurs only after 17 h (Table 3). Due to the slight unspecific cellular binding of the IgG modified doxorubicin loaded nanoparticles, as shown in FIG. 4C and FIG. 5C, the NP-Dox-IgG particles induce also cell death after 20 h. However, this NPDox-IgG induced cell death occurs later than with free doxorubicin incubation, which argues for a marginal unspecific doxorubicin uptake by the cells after NP-Dox-IgG incubation.

This NP-Dox-DI17E6 induced detachment and cellular apoptosis is further shown in a time lapsed acoustic microscopy movie in the supplement 1.

Cell viability assay: The biological activities of the different nanoparticulate formulations are tested in a MTT cell viability assay. The effectiveness of doxorubicin, either in free form or incorporated into nanoparticles, to reduce cell viability by 50% is expressed by IC-50 values (Table 4). NP-Dox-DI17E6 or non-PEGylated NP-Dox is more effective than free doxorubicin in αvβ3-positive M21 melanoma cells. Control nanoparticles coupled to an unspecific IgG mAb has no influence on cell viability in the tested concentrations (IC-50 value of NP-Dox 30.8±3.5 ng/ml, NP-Dox-DI17E6 8.0±0.2 ng/ml, free Doxorubicin 57.5±3.7 ng/ml, NP-Dox-IgG>100 ng/ml). In contrast, NP-Dox-DI17E6 does not reduce viability of αv-negative M21 L cells in the tested concentrations whereas free doxorubicin and non-PEGylated NP-Dox decreased M21L cell viability (IC-50 value of NP-Dox 75.4±8.3 ng/ml, NP-Dox-DI17E6 >100 ng/ml, free Doxorubicin 70.7±0.8 ng/ml, NP-Dox-IgG >100 ng/ml).

As used herein, the term “pharmaceutically acceptable” refers to compositions, carriers, diluents and reagents which represent materials that are capable of administration to or upon a mammal without the production of undesirable physiological effects such as nausea, dizziness, gastric upset and the like. The preparation of a pharmacological composition that contains active ingredients dissolved or dispersed therein is well understood in the art and need not be limited based on formulation. Typically, such compositions are prepared as injectables either as liquid solutions or suspensions, however, solid forms suitable for solution, or suspensions, in liquid prior to use can also be prepared. The preparation can also be emulsified. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. The therapeutic composition of the present invention can include pharmaceutically acceptable salts of the components therein.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin. vegetable oils such as cottonseed oil, and water-oil emulsions.

Typically, a therapeutically effective amount of an anti-integrin antibody according to the invention is an amount such that, when administered in physiologically tolerable composition, is sufficient to achieve a plasma concentration of from about 0.01 microgram (μg) per milliliter (ml) to about 100 μg/ml, preferably from about 1 μg/ml to about 5 μg/ml and usually about 5 μg/ml. Stated differently. the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily for one or several days. A preferred plasma concentration in molarity is from about 2 micromolar (μM) to about 5 millimolar (mM) and preferably, about 100 μM to 1 mM antibody antagonist.

The typical dosage of a chemical cytotoxic or chemotherapeutic agent according to the invention is 10 mg to 1000 mg, preferably about 20 to 200 mg, and more preferably 50 to 100 mg per kilogram body weight per day.

The pharmaceutical compositions of the invention can comprise phrase encompasses treatment of a subject with agents that reduce or avoid side effects associated with the combination therapy of the present invention (“adjunctive therapy”), including, but not limited to, those agents, for example, that reduce the toxic effect of anticancer drugs, e.g., bone resorption inhibitors, cardioprotective agents. Said adjunctive agents prevent or reduce the incidence of nausea and vomiting associated with chemotherapy, radiotherapy or operation, or reduce the incidence of infection associated with the administration of myelosuppressive anticancer drugs. Adjunctive agents are well known in the art. The immunotherapeutic agents according to the invention can additionally administered with adjuvants like BCG and immune system stimulators. Furthermore, the compositions may include immunotherapeutic agents or chemotherapeutic agents which contain cytotoxic effective radio-labeled isotopes, or other cytotoxic agents, such as a cytotoxic peptides (e.g. cytokines) or cytotoxic drugs and the like.

### FIGURES

FIG. 1 Thiolation of DI17E6 with a A.) 50 fold and B.) 100 fold molar excess of 2-iminothiolane. The antibody was analysed by size exclusion chromatography after 2, 5, 16, and 24 h of reaction time. DI17E6 was detected at a retention time of about 11 min whereas higher conjugates were detected at shorter retention times.

FIG. 2 Thiolation of DI17E6 for 2 h (black bars) and 5 h (hatched bars) with 5, 10, 50, or 100 molar excess of 2-iminothiolane, respectively. The amount of introduced thiol groups per antibody molecule was photometrically detected after reaction with Ellman's reagent (mean±SD; n=3).

FIG. 3: Proof of DI17E6 coupling on nanoparticle surface by scanning electron microscopy (SEM). Nanoparticles with DI17E6 coupling on surface (A, B=magnification of A in the red quadrangle) and nanoparticles without an antibody coupling (C) were incubated for 1 h at 4° C. with an 18 nm colloidal gold antihuman IgG antibody. The labelled nanoparticles were fixed and dehydrated. The examination was done with a SEM.

FIG. 4: Cellular binding of unloaded and doxorubicin loaded nanoparticulate formulations. αvβ3 integrin positive melanoma cells M21 (A and C) and αv-defective melanoma cells M21L (B and D) were treated with 2 ng/μl of the different unloaded (A and B) or doxorubicin loaded (C and D) nanoparticulate formulations for 4 h at 37° C. (concentrations are calculated referred to DI17E6 or equivalent NP amounts). Flow cytometry (FACS) analysis was performed to quantify their cellular binding. The data is shown as histogram of the FL1-H-channel (autofluorescence of the nanoparticles). Green: NP-DI17E6 and NP-Dox-DI17E6 respectively, red: NP-IgG and NP-Dox-IgG respectively, blue: untreated control. (ad A: one representative experiment out of 3 independent experiments is shown, ad B: n=1; ad C: one representative experiment out of 14 independent experiments is shown, ad D: n=1)

FIG. 5: Cellular uptake and intracellular distribution of nanoparticles studied by confocal laser scanning microscopy (CLSM). M21 cells were cultured on glass slides and treated with 10 ng/μl of the different nanoparticle formulations (referred to DI17E6 concentration or equivalent amount of control nanoparticles) for 4 h at 37° C. The green autofluorescence of the nanoparticles was used for detection and the red autofluorescence of doxorubicin. The cell membranes were stained with Concanavalin A AlexaFluor 350 (blue). Pictures were taken within inner sections of the cells. A): control, cells without nanoparticles, B) incubation of the cells with free doxorubicin, C) incubation of the cells with the unspecific nanoparticles with NP-Dox-IgG, D) incubation of the cells with the specific nanoparticles with NP-Dox-DI17E6.

FIG. 6: Cellular uptake and intracellular distribution of NP-Dox-DI17E6 studied by confocal laser scanning microscopy: split of the fluorescence channels. M21 cells were cultured on glass slides and treated with 10 ng/μl NP-Dox-DI17E6 for 4 h at 37° C. The green autofluorescence of the nanoparticles was used for detection and the red autofluorescence of doxorubicin. The cell membranes were stained with Concanavalin A AlexaFluor 350 (blue). Pictures were taken within inner sections of the cells. A): overlay of all fluorescence channels, B) display of the blue cell membrane channel, C) display of the green nanoparticles channel, D) display of the red doxorubicin channel.

FIG. 7: Cellular uptake and intracellular distribution of the NP-Dox-DI17E6 studied by confocal laser scanning microscopy: optical stack. M21 cells were cultured on glass slides and treated with 2 ng/μl NP-Dox-DI17E6 for 4 h at 37° C. The green autofluorescence of the nanoparticles was used for detection and the red autofluorescence of doxorubicin. The cell membranes were stained with Concanavalin A AlexaFluor 350 (blue). Cells were optically sliced in a stack of 1 μm thickness each and the picture series is displayed as a gallery.

FIG. 8: Cell attachment on vitronectin coated surface. 2 ng/μl of free DI17E6 or the different nanoparticulate formulations were incubated together with the αvβ3 integrin positive melanoma cells M21 on vitronectin coated ELISA plates (concentrations are calculated referred to DI17E6 or equivalent NP amounts). After 1 h of incubation non-adherent cells were removed. Remaining attached cells were stained with CyQUANT GR and counted against untreated control as described in the manufacturer's instructions manual. (Internal control of each experiment n=10, one representative experiment out of 3 independent experiments is shown.)

FIG. 9: Cell detachment from vitronectin coated surface. For cell detachment assay, 96-well ELISA plates were coated with vitronectin and cells were allowed to attach and spread for 1 h. Then, 4 ng/μl of free DI17E6 or the different unloaded or doxorubicin nanoparticulate formulations were added and the plates were incubated for additional 4 h at 37° C. to induce detachment (concentrations are calculated referred to DI17E6 or equivalent NP amounts). Detached cells were removed and remaining attached cells were stained with CyQUANT GR and counted against untreated control as described in the manufacturer's instructions manual. (Internal control of each experiment n=10, one representative experiment out of 9 independent experiments is shown.)

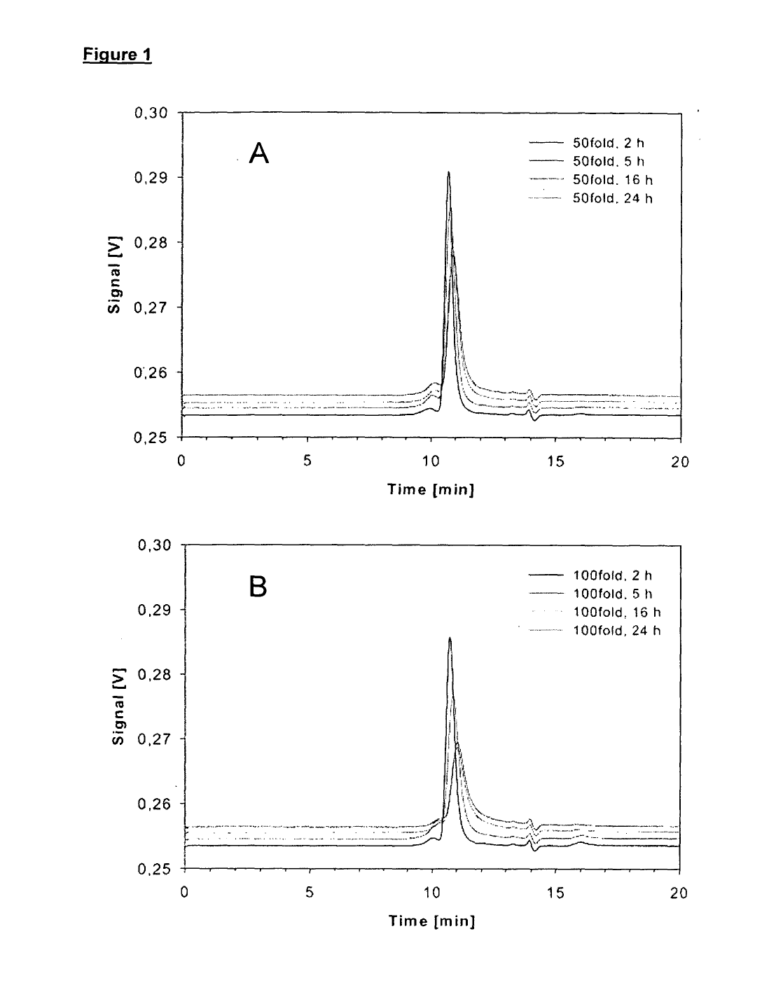

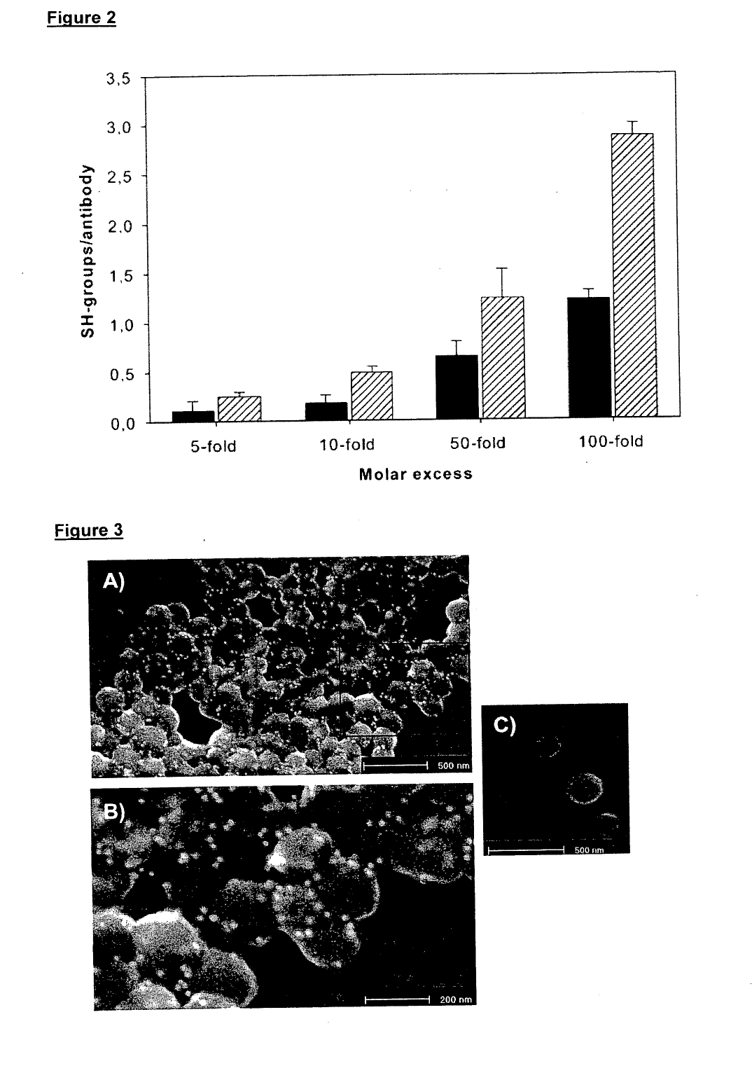

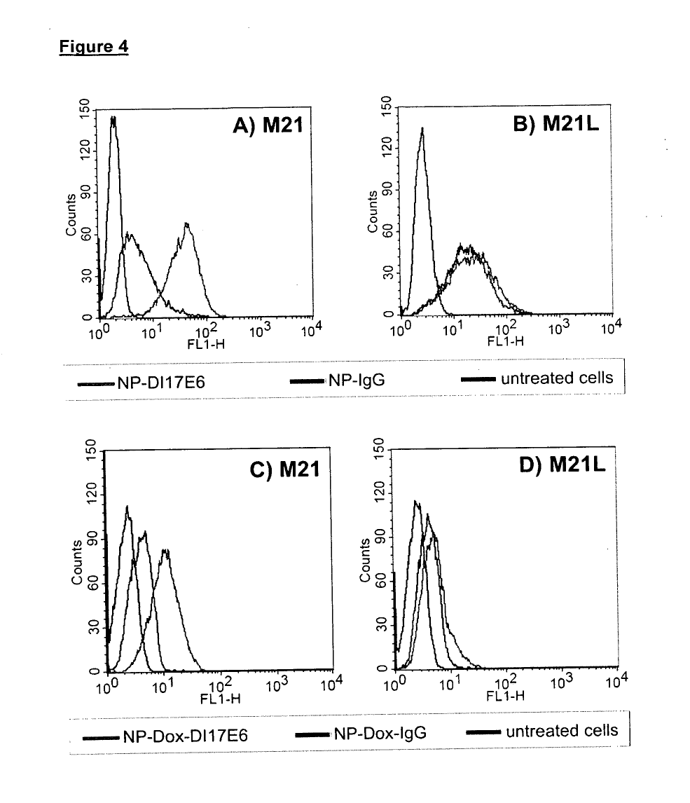

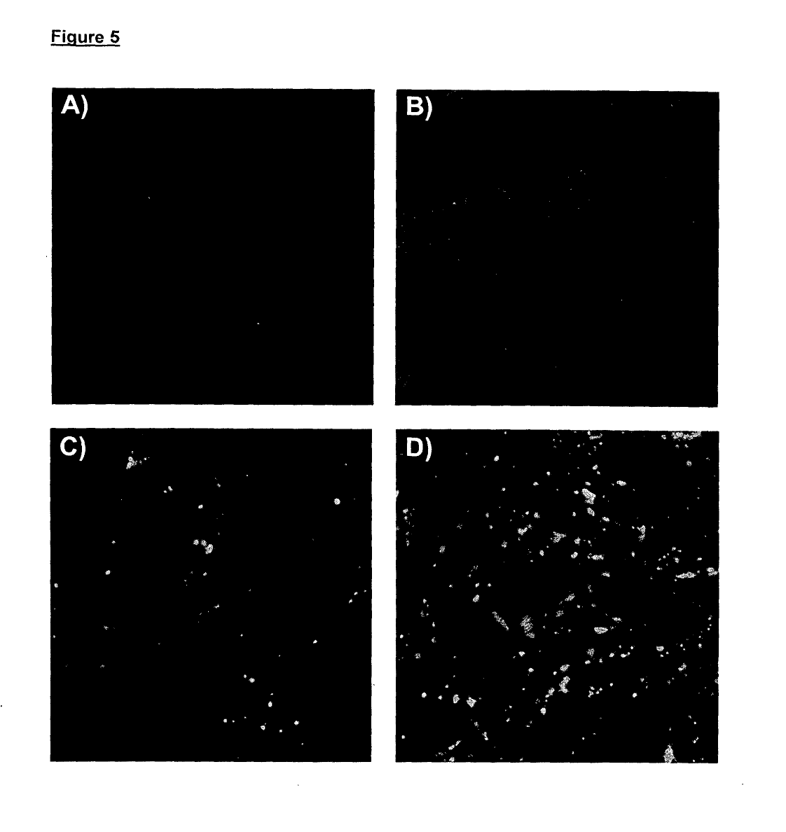

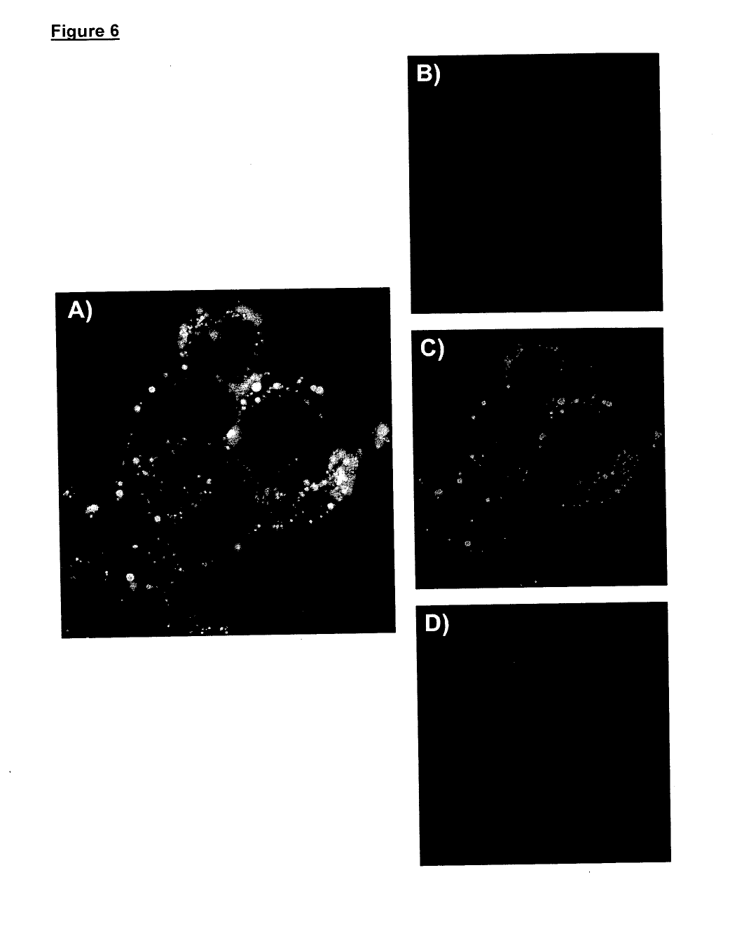

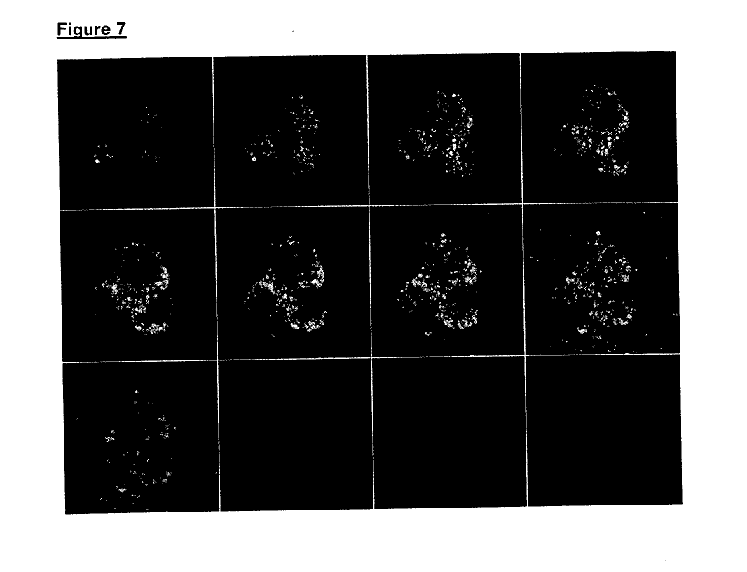

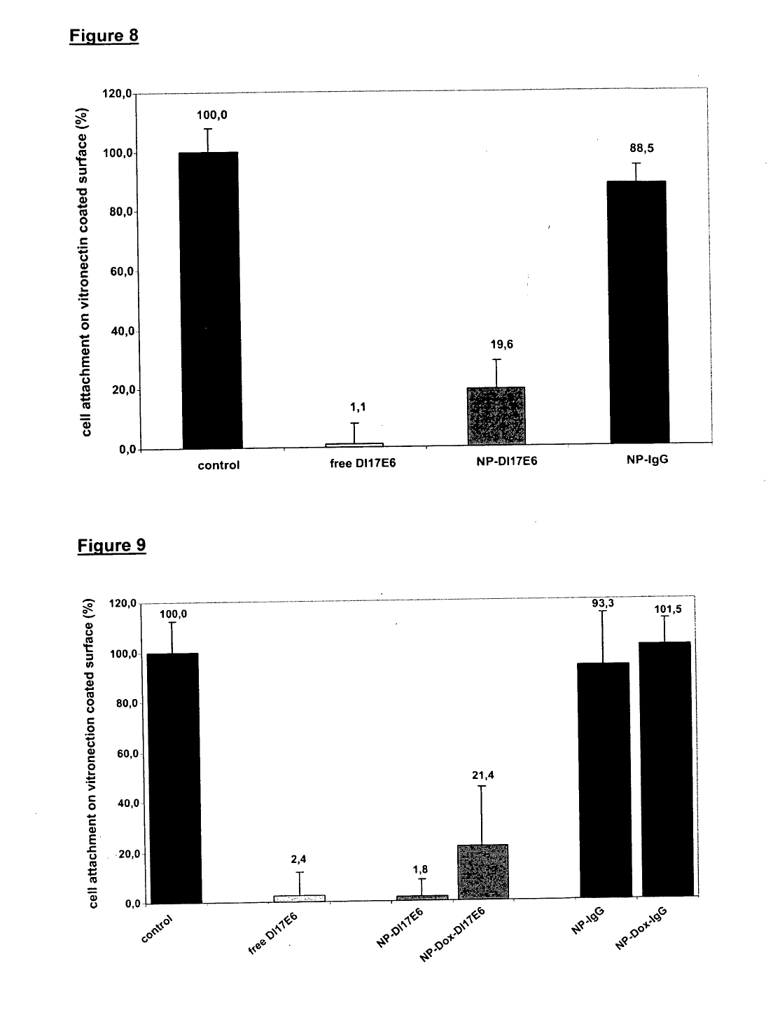

Supplement 1: Cell detachment from vitronectin coated surface: time lapsed acoustic microscopy As a further method to study the kinetics of cell detachment acoustic microscopy was used [41-43]. Therefore, αvβ3 integrin positive melanoma cells M21 were seeded on a vitronectin coated chamber, allowed to attach and spread and then incubated with doxorubicin loaded human serum albumin-nanoparticles with DI17E6-antibody coupling on particle surface. Detachment was observed by time lapsed acoustic microscopy over a period of 1-2 d. Pictures were done every minute. The detachment of the cells was analyzed by manual evaluation of the data.

### EXAMPLES

### Example 1

### Nanoparticle Preparation

(1) Reagents and chemicals: Human serum albumin (HSA, fraction V, purity 96-99%), glutaraldehyde 8% aqueous solution and human IgG antibody were obtained from Sigma (Steinheim, Germany). Doxorubicin was obtained from Sicor (Milan, Italy). 2-Iminothiolane (Traut's reagent), 5,5′-dithio-bis(2-nitro-benzoic acid) (Ellman's reagent) and D-Salt™ Dextran Desalting columns were purchased from Pierce (Rockford, USA), hydroxylamine hydrochloride and cysteine hydrochloride×H2O from Fluka (Buchs, Switzerland). DI17E6 was obtained from Merck KGaA, Darmstadt, Germany. The succinimidyl ester of methoxy poly(ethylene glycol) propionic acid with an average molecular weight of 5.0 kDa (mPEG5000-SPA) and the crosslinker poly(ethylene glycol)-α-maleimide-ω-NHS ester with an average molecular weight of 5.0 kDa (NHSPEG5000-Mal) were purchased from Nektar (Huntsville, USA). All reagents were of analytical grade and used as received.

(2) Thiolation of DI17E6: kinetics of dimerization reaction: Primary amino groups of the antibody can react with 2-iminothiolane, leading to introduction of sulfhydryl groups through ring opening reaction. Free sulfhydryl groups are necessary for subsequent covalent conjugation of the antibody via a linker to the particle surface. However, introduction of thiol groups bears the risk of oxidative disulfide bridge formation leading to dimers or even higher oligomers of DI17E6. DI17E6 was dissolved at a concentration of 1 mg/ml in phosphate buffer (pH 8.0). In order to introduce thiol groups 250.0 μl (50 fold molar excess) and 500.0 μl (100 fold molar excess) of 2-iminothiolane (6.9 mg in 50 ml phosphate buffer pH 8.0) were 6 added to 500.0 μl DI17E6 solution and the volume of the samples was adjusted with phosphate buffer (pH 8.0). These samples were incubated at 20° C. under constant shaking (600 rpm) for 2, 5, 16, or 24 h, respectively. The reaction was terminated by addition of 500.0 μl hydroxylamine solution (0.28 mg/ml in phosphate buffer, pH 8.0). This mixture was incubated for another 20 min. Afterwards, the samples were analyzed by size exclusion chromatography (SEC) on a SWXL column (7.8 mm×30 cm) in combination with a TSKgel SWXL guardcolumn (6 mm×4 cm) (Tosoh Bioscience, Stuttgart, Germany) using phosphate buffer (pH 6.6) as eluent at a flow rate of 1.0 ml/min to detect formation of di- or oligomers. Aliquots of 20.0 μl were injected and the eluent fraction was monitored by detection at 280 nm. In order to calibrate the SEC system for molecular weight, globular protein standards were used.

(3) Thiolation of DI17E6: quantification of thiol groups: DI17E6 was dissolved in phosphate buffer (pH 8.0) at a concentration of 1 mg/ml. This antibody solution (1000 μg/ml) was incubated with 4.02 μl (5 fold molar excess), 8.04 μl (10 fold molar excess), 40.2 μl (50 fold molar excess), or 80.4 μl (100 fold molars excess) of 2-iminothiolane solution (5.7 mg in 5.0 ml phosphate buffer, pH 8.0), respectively, for 2 h and 5 h at 20° C. under constant shaking. Using phosphate buffer as eluent the thiolated antibody was then purified by SEC using DSalt™ Dextran Desalting columns. The antibody containing fractions were detected photometrically at 280 nm and were pooled afterwards. The antibody solutions obtained from the purification step were concentrated to a content of about 1.1 mg/ml using Microcon® 30,000 microconcentrators (Amicon, Beverly, USA). Aliquots (250 μl) of concentrated DI17E6 solution were incubated with 6.25 μl Ellman's reagent (8.0 mg in 2.0 ml phosphate buffer pH 8.0) for 15 min at 25° C. Afterwards the samples were measured photometrically at 412 nm by using UVettes® (Eppendorf AG, Hamburg, Germany). In order to calculate the number of introduced thiol groups, L-cysteine standard solutions that were treated in the same way like the antibody solution were used. The content of DI17E6 was determined by microgravimetry.

(4) Preparation of unloaded nanoparticles: HSA (200 mg) was dissolved in 2 ml purified water. After filtration (0.22 μm) this solution was adjusted to pH 8.5. In order to form nanoparticles 8.0 ml ethanol were added at a rate of 1 ml/min by a tubing pump (Ismatec IPN, Glattbugg, Switzerland) under constant stirring at room temperature. The resulting particles were stabilized by using 8% glutaraldehyde solution (117.5 μl). The crosslinking process was performed for 24 h under constant stirring at room temperature. Particles were purified by two centrifugation steps (16,100 g, 10 min) and redispersed to original volume in phosphate buffer (pH 8.0). This redispersion was performed using a vortexer and ultrasonication.

(5) Preparation of doxorubicin-loaded nanoparticles 160 mg HSA were dissolved in 4 ml purified water and the solution was filtered through a 0.22 μm cellulose acetate membrane filter (Schleicher & Schuell, Dassel, Germany). An aliquot (500 μl) of this solution was added to 200 μl of a 0.5% (w/v) aqueous stock solution of doxorubicin. To this mixture, 300 μl of purified water were added. In order to adsorb doxorubicin to human serum albumin in solution, the mixture was incubated under stirring (550 rpm) for 2 h at room temperature. For the preparation of nanoparticles by desolvation, 3 ml ethanol (96%, v/v) were added continuously (1 ml/min) with a tubing pump (Ismatec IPN, Glattbrugg, Switzerland). After protein desolvation, an aliquot of 11.75 μl 8% glutaraldehyde solution was added to induce particle crosslinking (corresponding to 100% stoichiometric protein crosslinking). The crosslinking was performed for 24 h under constant stirring at ambient temperature. Aliquots (2.0 ml) of the resulting nanoparticles were purified by two cycles of differential centrifugation (16,100 g, 12 min) and redispersion. Within the first cycle redispersion was performed with 2.0 ml purified water whereas in the second cycle nanoparticles were redispersed with phosphate buffer (pH 8.0) to a volume of 500 μl using a vortexer and ultrasonication. The nanoparticle content was determined by gravimetry. The collected supernatants were used to determine the non-entrapped doxorubicin by HPLC. The content of entrapped doxorubicin was calculated from the difference between total doxorubicin and unbound drug. For the quantification of doxorubicin, a Merck Hitachi D7000 HPLC system equipped with a LiChroCART® 250-4 LiChrospher®-100 RP-18 column (Merck, Darmstadt, Germany) was used. Separation was obtained using a mobile phase of water and acetonitrile (70:30) containing 0.1% trifluoroacetic acid at a flow rate of 0.8 ml/min. Doxorubicin was quantified by UV (250 nm) and fluorescence detection (excitation 560 nm, emission 650 nm).

(6) Surface modification of nanoparticles: Unloaded and drug loaded HSA nanoparticles were prepared as described earlier and were modified as follows: One milliliter of HSA nanoparticle suspension dispersed in phosphate buffer (pH 8.0) was incubated with 250 μl of mPEG5000-SPA solution (60 mg/ml in phosphate buffer pH 8.0) or poly(ethylene glycol)-α-maleimide-ω-NHS ester, respectively, for 1 h at 20° C. under constant shaking (Eppendorf thermomixer, 600 rpm). The nanoparticles were purified by centrifugation and redispersion as described above. The content of the nanoparticles was determined by microgravimetry.

For the thiolation step of the antibodies, DI17E6 or IgG were dissolved in phosphate buffer pH 8.0 at a concentration of 1.0 mg/ml. For the introduction of thiol groups DI17E6 or IgG, respectively, were incubated with a 50 fold molar excess of 2-iminothiolane solution (c=1.14 mg/ml; 40.2 μl) for 2 h as previously described by Steinhauser et al. (2006) [7]. The antibodies were purified by size exclusion chromatography (SEC, D-Salt™ Dextran Desalting column). The resulting solutions contained thiolated antibody (DI17E6 or IgG, respectively) at a concentration of about 500 μg/ml. For the coupling reaction 1.0 ml of the sulfhydryl-reactive nanoparticle suspension was incubated with 1.0 ml of the thiolated DI17E6 or IgG, respectively, to achieve a covalent linkage between antibody and the nanoparticle system. For the preparation of samples with adsorptively attached antibody, 1.0 ml of the mPEG5000-SPA modified nanoparticles were incubated with 1.0 ml of thiolated DI17E6 or IgG, respectively. The incubation of all samples was performed for 12 h at 20° C. under constant shaking (600 rpm). The samples were purified from unreacted antibody by centrifugation and redispersion as described earlier. To determine unbound antibody the resulting supernatants were collected and analyzed by size exclusion chromatography (SEC) as described above. The amount of antibody bound to the nanoparticle surface was calculated as difference between the amount of antibody obtained after thiolation and purification and the amount of antibody determined in the supernatant obtained after the conjugation step.

### Example 2

### Nanoparticle Characterization

Nanoparticles were analyzed with regard to particle diameter and polydispersity by photon correlation spectroscopy (PCS) using a Malvern Zetasizer 3000HSA (Malvern Instruments Ltd., Malvern, UK). The zetapotential was measured with the same instrument by Laser Doppler microelectrophoresis. Prior to both measurements the samples were diluted with filtered (0.22 μm) purified water. Particle content was determined by microgravimetry. For this purpose 50.0 μl of the nanoparticle suspension was pipetted into an aluminium weighing dish and dried for 2 h at 80° C. After 30 min of storage in an exsiccator the samples were weighed on a micro balance (Sartorius, Germany).

### Example 3

### Proof of Antibody Coupling on Nanoparticle Surface

Nanoparticles with DI17E6 coupling on surface (NP-DI17E6) and nanoparticles without antibody coupling (NP) were incubated for 1 h at 4° C. with an 18 nm colloidal gold anti-human IgG antibody (dianova, Hamburg, Germany) in PBS. The labeled nanoparticles were fixed with 2% glutaraldehyde in 0.1 M sodium cacodylate buffer, filtered through a Millipore filter (0.22 μm) or Millipore Filter inserts. Then the samples were dehydrated in 30%, 50%, and 100% ethanol, air-dried, coated with carbon in a SCD-030 coater (Balzers, Liechtenstein) and examined in a field emission scanning electron microscope FESEM XL30 (Phillips, USA). An accelerating voltage of 10 kV was used for secondary electron (SE) imaging. For detection of the antibody on the nanoparticle surface the samples were studied using backscattered electron (BSE) modes.

### Example 4

### Cell Culture

The αvβ3 integrin positive melanoma cell line M21 was used for all experiments. The αv-negative melanoma cell line M21 L was used as control (both cell lines provided by Merck KGaA).

The cells were cultured at 37° C. and 5% CO2 in RPMI1640 medium (Invitrogen, Karlsruhe, Germany) supplemented with 10% fetal calf serum (Invitrogen, Karlsruhe, Germany), 1% pyruvate (Invitrogen, Karlsruhe, Germany) and antibiotics (50 U/ml penicillin and 50 μg/ml streptomycin; Invitrogen, Karlsruhe, Germany). The PBS contained Ca2+/Mg2+ (Invitrogen, Karlsruhe, Germany).

### Example 5

### Cellular Binding

M21 or M21L cells were cultured in 24-well plates (Greiner, Frickenhausen, Germany) and treated with the different nanoparticle formulations for 4 h at 37° C. For the testing of DI17E6 modified nanoparticles, concentrations of 2 ng/μl, referred to DI17E6 concentration coupled on the particle surface, were employed. Control nanoparticles without DI17E6 modification were used in equivalent nanoparticle quantities. After incubation, cells were washed twice with PBS (Invitrogen, Karlsruhe, Germany), then trypsinized and harvested. After fixing with FACS-Fix (10 g/l PFA and 8.5 g/l NaCl in PBS, pH 7.4), flow cytometry (FACS) analysis was performed with 10,000 cells per sample, using FACSCalibur and CellQuest Pro software (Becton Dickinson, Heidelberg, Germany). Nanoparticles could be detected at 488/520 nm.

### Example 6

### Cellular Uptake and Intracellular Distribution

Cellular uptake and intracellular distribution of the nanoparticles were studied by confocal laser scanning microscopy. M21 cells were cultured on glass slides and treated with 2 ng/μl or 10 ng/μl of the different nanoparticle formulations for 4 h at 37° C. (concentrations are calculated referred to DI17E6 or equivalent NP amounts as described in 2.5). After the incubation period, cells were washed twice with PBS and cell membranes were stained with 50 ng/μl Concanavalin A AlexaFluor 350 (346/442° nm) (Invitrogen, Karlsruhe, Germany) for 2 min. Cells were fixed with 0.5% PFA for 5 min. After fixation, cells were washed and embedded in Vectashield HardSet Mounting Medium (Axxora, Grünberg, Germany). The confocal microscopy study was performed with an Axiovert 200M microscope with a 510 NLO Meta device (Zeiss, Jena, Germany), MaiTai femtosecond or an argon ion laser and the LSM Image Examiner software. Nanoparticles were detected at 488/520 nm. Doxorubicin was detected by red fluorescence at 488/590 nm.

### Example 7

### Cell Attachment and Detachment Assay

αvβ3 integrin positive melanoma cells M21 were grown on vitronectin (MoBiTec, Göttingen, Germany) coated ELISA plates (Nunc, Wiesbaden, Germany). Therefore, ELISA 96-well plates were coated with 1 μg/ml vitronectin for 1 h at 37° C. Plates were blocked with 1% heat inactivated BSA (PAA, Cölbe, Germany) and incubated with either 2 ng/μl of free DI17E6 or the different nanoparticulate formulations (referred to free mAb) together with the cells in cell adhesion medium (RPMI 1640 with 2 mM L-glutamine supplemented with 1% BSA). After 1 h of incubation at 37° C., non-adherent cells were removed by gentle washing with prewarmed PBS. Remaining attached cells were stained with CyQUANT GR (Invitrogen, Karlsruhe) and counted against untreated control in a microtiter ELISA reader as described in the manufacturer instructions manual.

For cell detachment assays, 96-well ELISA plates were coated with vitronectin as described above. After blocking, cells were allowed to attach and spread for 1 h in cell adhesion medium. Then, 4 ng/μl or 10 ng/μl of either free DI17E6 or the different nanoparticulate formulations (referred to free mAb) were added and the plates were incubated for additional 4 h at 37° C. to induce detachment. Subsequently, plates were washed and processed as for cell adhesion assay. Specific inhibition of attachment or induction of detachment were determined relative to vitronectin-coated surfaces blocked with BSA.

### Example 8

### Kinetic of Cell Detachment

For the determination of cell detachment kinetics, cells were seeded in a vitronectin coated multiwell chamber and incubated with the different nanoparticulate formulations or free doxorubicin in a humidified, CO2-aerated climate chamber at 37° C. Detachment was observed by transmitted light time lapse microscopy over a period of 1-2 d. Pictures were done every 7 minutes. The detachment of the cells was analyzed by manual evaluation of the data.

### Example 9

### Cell Viability Assay

Cell viability was assessed using the 3-(4,5-dimethylthiazol-2-yl)-2,5-diphenyltetrazolium bromide (MTT) dye reduction assay [27] modified as described before [28].

TABLE 1
Physico-chemical characteristics of DI17E6 and IgG modified HSA nanoparticles with 100%
crosslinking (mean ± SD; n = 3).

| HSA- | | | | | | |
|---|---|---|---|---|---|---|
| nanoparticles | | | Covalent | Covalent | Adsorptive | Adsorptive |
| 100% | | | binding | binding | binding | binding |
| crosslinking | | Unmodified | of DI17E6 | of IgG | of DI17E6 | of IgG |
| Particle | [nm] | 166.5 ± 17.6 | 181.4 ± 16.4 | 181.6 ± 15.6 | 172.8 ± 14.5 | 172.0 ± 14.7 |
| diameter | | | | | | |
| Polydispersity | | 0.034 ± 0.012 | 0.026 ± 0.013 | 0.063 ± 0.045 | 0.011 ± 0.009 | 0.024 ± 0.018 |
| Zetapotential | [mV] | −43.3 ± 1.1 | −37.4 ± 2.9 | −38.4 ± 0.7 | −39.7 ± 1.4 | −39.2 ± 2.4 |
| Particle | [mg/ml] | 19.42 ± 1.62 | 15.92 ± 0.60 | 16.02 ± 1.99 | 16.65 ± 0.94 | 16.68 ± 1.03 |
| content | | | | | | |
| Antibody | [μg/mg] | | 16.10 ± 1.90 | 16.78 ± 0.47 | 2.63 ± 1.32 | 6.12 ± 2.03 |
| binding | | | | | | |
| efficiency | | | | | | |

TABLE 2
Physico-chemical characteristics of DI17E6 and IgG modified doxorubicin-loaded HSA nanoparticles
with 100% crosslinking (mean ± SD; n = 3)

| Doxorubicin- | | | | | | |
|---|---|---|---|---|---|---|
| loaded HSA- | | | | | | |
| nanoparticles | | | Covalent | Covalent | Adsorptive | Adsorptive |
| 100% | | | binding | binding | binding | binding |
| crosslinking | | Unmodified | of DI17E6 | of IgG | of DI17E6 | of IgG |
| Particle | [nm] | 379.5 ± 21.5 | 404.9 ± 27.0 | 406.1 ± 35.8 | 391.0 ± 23.2 | 386.5 ± 24.9 |
| diameter | | | | | | |
| Polydispersity | | 0.086 ± 0.025 | 0.040 ± 0.045 | 0.036 ± 0.021 | 0.054 ± 0.025 | 0.043 ± 0.034 |
| Zetapotential | [mV] | −33.1 ± 2.6 | −40.3 ± 3.1 | −39.1 ± 4.2 | −41.4 ± 5.4 | −37.0 ± 7.1 |
| Particle | [mg/ml] | 15.3 ± 1.1 | 14.4 ± 1.2 | 14.4 ± 1.1 | 14.7 ± 1.1 | 14.8 ± 1.3 |
| content | | | | | | |
| Antibody | [μg/mg] | | 15.84 ± 4.07 | 17.31 ± 2.37 | 0.16 ± 0.28 | 2.95 ± 0.56 |
| binding | | | | | | |
| efficiency | | | | | | |
| Drug loading | [μg/mg] | 56.7 ± 2.9 | 56.7 ± 2.9 | 56.7 ± 2.9 | 56.7 ± 2.9 | 56.7 ± 2.9 |

TABLE 3
Calculation of time-lapsed detachment measurement

| | Detachment | Cell death |
|---|---|---|
| Sample | [h after incubation *] | [h after incubation *] |
| NP-Dox-DI17E6 | 0.25-3 | 10 |
| NP-DI17E6 | 2-22 | — |
| free doxorubicin | — | 17 |
| NP-Dox-IgG | — | 20 |
| control | — | — |

* Total incubation time: 1-2 d

TABLE 4
IC-50 values of different nanoparticulate formulations

| | M21 | M21L |
|---|---|---|
| | [ng/ml] | [ng/ml] |
| Nanoparticle preparation | | |
| NP-Dox unmodified | 30.8 ± 3.5 | 75.4 ± 8.3 |
| NP-Dox-Peg | >100 | >100 |
| NP-Dox-DI17E6 | 8.0 ± 0.2 | >100 |
| NP-Dox-IgG | >100 | >100 |
| Controls | | |
| free doxorubicin | 57.5 ± 3.7 | 70.7 ± 0.8 |
| free DI17E6 | >100 | >100 |

1. An anti-integrin antibody nanoparticle conjugate, obtained by linking covalently an anti-integrin antibody or a biologically active fragment thereof to the surface of a protein-nanoparticle which was prior treated with a chemotherapeutic agent.

2. An antibody nanoparticle conjugate of claim 1, wherein the chemotherapeutic agent was loaded by adsorption to the protein-nanoparticle.

3. An antibody nanoparticle conjugate of claim 1, wherein the protein nanoparticle is of human serum albumin (HSA) or bovine serum albumin (BSA).

4. An antibody nanoparticle conjugate of claim 1, wherein the particle diameter of the untreated protein-nanoparticles is between 150 and 280 nm.

5. An antibody nanoparticle conjugate of claim 1, wherein the particle diameter of the protein-nanoparticles treated with a chemotherapeutic agent is between 300 and 390 nm.

6. An antibody nanoparticle conjugate of claim 1, wherein the antibody was linked directly or by a linker to the protein-nanoparticle via a sulfhydryl group introduced into the antibody molecule.

7. An antibody nanoparticle conjugate of claim 1, wherein the chemotherapeutic agent treated with said protein-nanoparticle is selected from the group consisting of: cisplatin, doxorubicin, gemcitabine, docetaxel, paclitaxel, bleomycin and irinotecan.

8. An antibody nanoparticle conjugate of claim 1, wherein the antibody linked covalently to said protein-nanoparticle is selected from the group LM609, vitaxin, and 17E6 and variants thereof.

9. An antibody nanoparticle conjugate of claim 1, wherein the protein-nanoparticle is HSA that is loaded with doxorubicin and the antibody linked covalently to this particle is 17E6 or DI17E6.

10. A pharmaceutical composition comprising an antibody nanoparticle conjugate as specified in claim 1 in an pharmacologically effective amount optionally together with a pharmacologically acceptable carrier, eluent or recipient.

11. Use of an antibody nanoparticle conjugate as specified in claim 1 for the manufacture of a medicament for the treatment of cancer diseases.

12. An antibody nanoparticle conjugate as specified in claim 1 for use in the treatment of tumor diseases.
